(19) United States
(12) Patent Application Publication
(10) Pub. No.: US 2006/0252150 A1
(43) Pub. Date: Nov. 9, 2006

(54) Human embryonic stem cell cultures, and compositions and methods for growing same

(76) Inventor: Linzhao Cheng, Columbia, MD (US)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 10/533,514

(22) PCT Filed: Nov. 10, 2003
(86) PCT No.: PCT/US03/35734

§ 371 Date: Jan. 23, 2006

Related U.S. Application Data
(60) Provisional application No. 60/425,228, filed on Nov. 8, 2002.

(51) Int. Cl. C12N 5/08

(52) U.S. Cl. 435/372

(57) ABSTRACT

Human pluripotential embryonic stem cell cultures are provided, as are human feeder cells useful for growing the human embryonic stem cells, conditioned medium obtained from cultures of the human feeder cells, and factors derived from the conditioned medium. Also provided are methods of growing human embryonic stem cells in the presence of the human feeder cells, the conditioned medium, the factors derived from the conditioned medium, or a combination thereof. In addition to the human embryonic stem cell cultures grown according to such methods, isolated human embryonic stem cells obtained from such human embryonic stem cell cultures are provided, as are methods of using such isolated cells.

### CROSS REFERENCE TO RELATED APPLICATION

This application claims the benefit of priority under 35 U.S.C. § 119(e) of U.S. Ser. No. 60/425,228, filed Nov. 8, 2002, which is incorporated herein by reference.

### GRANT INFORMATION

This invention was made with government support under Grant No. P30 CA 06973 awarded by the National Cancer Institute. The United States government has certain rights in this invention.

### BACKGROUND OF THE INVENTION

1. Field of the Invention

The invention relates generally to human cell cultures and more specifically to human feeder cells and factors derived therefrom, which are useful for growing human embryonic stem cells, methods of growing human embryonic stem cells, human embryonic stem cell cultures, and isolated human embryonic cells derived from such cultures.

2. Background Information

Stem cells are precursor cells that, upon differentiation, give rise to all cells in an adult organism, including a human. Embryonic stem (ES) cells are pluripotential undifferentiated cells that can differentiate into any cell type, including muscle cells, bone cells, neuronal cells, blood cells, liver cells, pancreatic cells, etc. (see, e.g., at hypertext transfer protocol (“http”), on the world wide web (“www”), URL “news.wisc.edu/packages/stemcells”). ES cells also can differentiate into multipotential stem cells, which are relatively undifferentiated cells that are destined to give rise to specific cell types, e.g., blood cells.

Human ES (hES) cells can be derived fertilized embryos that are less than one week old, e.g., embryos obtained by in vitro fertilization. Several hES cell lines have been established, and are available from specified sources (e.g., WiCell Research Institute, Inc., a non-profit organization that has been designated a National Stem Cell Center; see, at “http”, on the “www”, URL “wicell.org/index.jsp”). Because they can differentiate into any adult cell type, hES cells provide the promise of hope for treating many diseases and disorders, including degenerative conditions associated with a disease (e.g., a neurodegenerative disease such as Parkinson's disease or a musculodegenenerative disease such as muscular dystrophy) and conditions having a congenital basis or associated with aging or injury (e.g., deafness and spinal cord injuries).

Human ES cells also provide a tool for drug screening assays because they provide a means to obtain pure populations of specific cell types, including cell types associated with specific disorders. As such, the hES cells, or pure populations of cells derived from hES cells, can be used in screening assays to identify drugs that can affect the particular cell type in a manner indicating that the drug can be useful for treating a disorder associated with the cell type (see URL “news.wisc.edu/packages/stemcells, supra).

In order for hES cell therapy to reach its full potential, convenient and ethically acceptable ways for obtaining large numbers of the hES cells are required. Currently, prolonged propagation of hES cells has been achieved by co-culturing the hES cells with primary mouse embryonic fibroblasts (pMEFs), which serve as feeder cells. However, the requirement that undifferentiated hES cells be co-cultured with pMEFs has impeded clinical applications because of the risk associated with administering to human patients hES cells that have been in contact with uncharacterized rodent cells. Human fetal cells also have been reported to be useful as feeder cells for culturing hES cells. However, while human fetal cells obviate potential problems associated with rodent cells, the use of fetal cells presents ethical issues that can be difficult to resolve. Thus, a need exists for methods of growing undifferentiated pluripotential hES cells such that the hES cells, and cells derived therefrom, are clinically and socially acceptable for administration to human individuals.

### SUMMARY OF THE INVENTION

The present invention is based on the determination that adult human cells can be used as feeder cells for growing continuous cultures of undifferentiated pluripotential human embryonic stem (hES) cells. Adult human bone marrow stromal cells (hMSCs) and adult human fibroblasts derived from breast skin, as well as hMSCs and human fibroblasts immortalized by transduction with a human telomerase gene, and conditioned medium from such cells, supported hES cell growth in culture. Remarkably, the hES cells passaged in culture using the disclosed compositions and methods have maintained a diploid karyotype and have remained in an undifferentiated state after continuous culture and many passages. The availability of adult human feeder cells, and compositions derived therefrom, provide an animal cell-free and serum-free system for obtaining clinically useful numbers of hES cells in an ethically acceptable manner.

Accordingly, the present invention relates to isolated undifferentiated pluripotential human embryonic stem (hES) cells, wherein the hES cells exhibit dependence on adult human feeder cells, or an hES cell-maintaining product of said adult human feeder cells, for maintenance in culture. Such hES cells are distinguishable from hES cells that have been passaged using rodent cell feeder cells in that they are free of potentially contaminating rodent viruses and other materials associated with or produced by rodent cells in culture. Adult human feeder cells can be any adult human cells that support growth and proliferation of hES cells, and that maintain the hES cells in an undifferentiated pluripotential state. Such adult human feeder cells are exemplified herein by human bone marrow stromal cells, and by human fibroblasts such as CCD-1087sk cells, which are derived from human breast skin. As disclosed herein, the adult human feeder cells can be immortalized cells, thus providing a continuous and standard source of the feeder cells. Adult human feeder cells can be immortalized, for example, by expressing an exogenous polynucleotide encoding a telomerase in the feeder cells.

Undifferentiated pluripotential hES cells can be maintained and grown by co-culturing the hES cells with adult human feeder cells; by culturing the hES cells in conditioned medium, which can be obtained by culturing adult human feeder cells in a growth medium (e.g., a minimal growth medium) and collecting the medium; or by culturing the hES cells in an isolated fraction of such conditioned medium, wherein the fraction contains biomolecules having a molecular weight of about 30 kiloDaltons (kDa) and greater (e.g., an enriched fraction of conditioned medium obtained using standard fractionation methods such as centrifugal filtration).

The present invention also relates to a culture of undifferentiated pluripotential hES cells. Such a culture can contain, in addition to the hES cells, supportive adult human feeder cells, or a product of such feeder cells that allows continuous passage of the hES cells. Such a product can be, for example, conditioned medium obtained from a culture of the supportive adult human feeder cells, or an enriched fraction of the conditioned medium containing biomolecules having a molecular mass of about 30 kDa and greater, which can maintain and allow proliferation of hES cells. In one embodiment, the culture contains hES cells and supportive adult human feeder cells, which can, but need not, be immortalized feeder cells, and can, but need not, be irradiated such that the feeder cells are alive but incapable of proliferation. Examples of supportive adult human feeder cells include human bone marrow stromal cells and CCD-1087sk fibroblasts, which are derived from breast skin.

In another embodiment, the culture contains hES cells and an hES cell-maintaining product produced by supportive adult human feeder cells. Such an hES cell-maintaining product of supportive adult human feeder cells can be conditioned medium that is produced upon culture of the feeder cells in a growth medium; or can be a fraction of such conditioned medium, particularly a fraction containing biomolecules having a molecular mass greater than about 30 kDa, including biological molecules produced by the adult human feeder cells. In one aspect of this embodiment, the culture further contains non-supportive feeder cells, which, alone, cannot support hES cell growth but which, in combination with an hES cell-maintaining product of supportive adult human feeder cells, can maintain the hES cell culture. Such non-supportive feeder cells can, but need not, be human cells.

The present invention further relates to a method of obtaining an expanded population of undifferentiated pluripotential hES cells. Such a method can be performed, for example, by culturing hES cells with supportive adult human feeder cells, or by culturing hES cells with an hES cell-maintaining product of supportive adult feeder cells, under conditions suitable for growth of the hES cells. As such, the invention provides a culture of undifferentiated pluripotential hES cells prepared by such a method.

According to a method of the invention, the supportive adult human feeder cells can be any adult human cells that produce biomolecules that support the growth and proliferation of hES cells in culture. Such supportive adult human feeder cells are exemplified by human bone marrow stromal cells and by adult breast skin fibroblasts (CCD-1087sk cells). Such cells, either alone or in combination with each other or with other cells can be used in a method of the invention. The supportive adult human feeder cells can, but need not, be immortalized, such that they are amenable to long term and continuous culture.

A method of obtaining an expanded population of undifferentiated pluripotential hES cells can further include a step of isolating hES cells of the expanded population, thus providing isolated undifferentiated pluripotential hES cells. Accordingly, isolated undifferentiated pluripotential hES cells obtained by such a method are provided. In addition, a method of obtaining an expanded population of undifferentiated pluripotential hES cells can include one or more steps of sub-culturing hES cells of the expanded population of hES cells under conditions suitable for growth, including, as desired, conditions suitable for growth of undifferentiated pluripotential hES cells. As such, the methods provide a means to obtain one or more sub-cultures of hES cells, or cells derived therefrom, including hES cells at various passages in culture, and provide a means to obtain a continuous culture of undifferentiated pluripotential hES cells. Accordingly, populations of hES cells at different passages in culture are provided, as are continuous cultures of undifferentiated pluripotential hES cells.

A method of obtaining an expanded population of undifferentiated pluripotential hES cells, including a method encompassing sub-culturing the hES cells, also can include a step of freezing one or more aliquots of the expanded and/or sub-cultured population of hES cells. The hES cells can be frozen such that they can be used as a source of proteins, nucleic acids, or the like specific for the hES cells, or can be frozen under conditions such that the hES cells remain viable, thus providing hES cells that can be stored frozen for future use or for conveniently disseminating the hES cells to others. Accordingly, the invention also provides at least one aliquot of frozen undifferentiated pluripotent hES cells obtained by such a method, and further provides a plurality of aliquots of frozen undifferentiated pluripotent hES cells, wherein, for example, two or more aliquots of the plurality contain hES cells of the same or different passage numbers. In one aspect, the aliquots of frozen cells are viable and, in a further aspect, can be cultured upon thawing.

A method of obtaining an expanded population of undifferentiated pluripotential hES cells also can include a step of inducing differentiation of hES cells of the expanded population, thereby obtaining a population of differentiated cells. Accordingly, the invention further provides a population of differentiated cells obtained by such a method. Such a method provides the advantage that a substantially pure population of differentiated cells can be obtained, thus providing a means to obtain substantially one cell type without contamination by other cell types. Such substantially pure populations of differentiated cells, which can be multipotential cells or terminally differentiated cells, can be used, for example, in screening assays and for therapeutic purposes.

The present invention also relates to a method for identifying an agent that alters a function of an undifferentiated pluripotential hES cell. Such a method can be performed, for example, by contacting hES cells with a test agent, wherein the hES cells exhibit dependence on adult human feeder cells, or an hES cell-maintaining product of said adult human feeder cells, for maintenance in culture; and detecting a change in a function of the hES cells in presence of the test agent as compared to the function in the absence of the test agent, thereby identifying the test agent as an agent that alters the function of the hES cells. Such a method can be performed by contacting the test agent and hES cells in vivo, for example, following administration or implantation of the hES cells into a subject, or by contacting the test agent an hES cells in vitro, for example, by adding the test agent to a culture containing the hES cells or to hES cells isolated from a culture.

The function of undifferentiated pluripotential hES cell that can be altered due to contact with an agent can be any function of the hES cells. For example, the function can be expression of gene that typically is expressed (or not expressed) in hES cells, and the agent can alter the function by increasing or decreasing the level of expression of an expressed gene (e.g., decreasing expression of stage-specific surface antigen-4, alkaline phosphatase, or Oct-4 transcription factor), or by turning on the expression of an unexpressed gene (e.g., inducing expression of stage-specific surface antigen-1), in the hES cells. In one embodiment, the agent that effects a function of hES cells is one that induces differentiation of the hES cells, thereby producing differentiated cells. Such differentiated cells can be multipotential human stem cells (e.g., hematopoietic stem cells) or can be terminally differentiated cells (e.g., muscle cells, neuronal cells, blood cells, connective tissue, or epithelial cells). As such, the method can be used to identify an agent that induces differentiation of hES cell to pancreatic beta cells, hepatocytes, cardiomyocytes, skeletal muscle cells, or any other cell type.

The present invention further relates to a method of obtaining biomolecules that are required for growth of undifferentiated pluripotential hES cells in culture. Such a method can be performed, for example, by culturing adult human cells that can support the growth of hES cells in culture; and isolating conditioned medium generated by culturing the adult human cells, wherein the condition medium contains biomolecules that support hES cell growth in culture. The adult human cells that can support the growth of hES cells in culture can be, for example, human bone marrow stromal cells or human fibroblasts such as CCD-1087sk human breast skin fibroblasts. Such a method can further include obtaining from the conditioned medium an enriched fraction containing biomolecules having a molecular mass greater than about 30 kDa, which, as disclosed herein, can support the growth of undifferentiated pluripotent hES cells. Such an enriched fraction can be obtained, for example, by collecting a gel chromatography fraction such as the flow through fraction from a column that excludes material having a molecular mass greater than about 30 kDa, or by a centrifugal filtration method using a filter with an appropriate nominal molecule weight limit. Accordingly, the invention provides conditioned medium obtained by a method of the invention, wherein the conditioned medium supports undifferentiated pluripotential hES cell growth. Also provided is an enriched fraction of such conditioned medium that supports undifferentiated pluripotential hES cell growth, wherein the enriched fraction contains biomolecules having a molecular mass greater than about 30 kDa.

The present invention also relates to a method for obtaining undifferentiated pluripotential hES cells. Such a method can be performed, for example, by culturing a suspension of cells that includes undifferentiated pluripotential hES cells, and supportive adult human feeder cells (or an hES cell-maintaining product of said feeder cells), under conditions suitable for growth of the hES cells; and isolating cells that express SSEA-4, Oct-4, and alkaline phosphatase, and do not express SSEA-1. Accordingly, isolated undifferentiated pluripotential hES cells obtained by such a method also are provided.

The suspension of cells comprising undifferentiated pluripotential hES cells can be a suspension of cells such as those available from a National Stem Cell Center, or can be a cell suspension prepared from an embryo that is less than about one week old, for example, an embryo obtained by in vitro fertilization. In one embodiment, the method for obtaining undifferentiated pluripotential hES cells is performed by culturing the suspension comprising hES cells and the supportive adult human feeder cells (e.g., human bone marrow stromal cells). In another embodiment, the method is performed by culturing the suspension comprising hES cells, an hES cell-maintaining product of the supportive adult human feeder cells (e.g., conditioned medium), and, optionally, non-supportive feeder cells (i.e., cells that, alone, cannot support hES cell growth). Preferably, the non-supportive feeder cells are human cells (e.g., adult human cells).

The present invention further relates to a method of ameliorating a pathologic condition in a subject. Such a method can be performed, for example, by administering undifferentiated pluripotential hES cells, which are cultured as disclosed herein, or cells derived from said hES cells, to the subject, wherein the hES cells exhibit dependence on adult human feeder cells, or an hES cell-maintaining product of said adult human feeder cells, for maintenance in culture. The pathologic condition to be treated according to such a method can be any condition amenable to treatment using the hES cells or differentiated cells derived from the hES cells. Accordingly, the condition can be a degenerative disorder (e.g., Parkinson's disease, Alzheimer's disease, macular degeneration, or muscular dystrophy), an autoimmune disorder (e.g., multiple sclerosis), or other disorder such as diabetes or kidney disease. The pathologic condition also can be the result of an injury, for example, a spinal cord injury, a burn, a stroke, or a myocardial infarction.

### BRIEF DESCRIPTION OF THE DRAWING

FIG. 1 shows the number of hES cell colonies following co-culture with pMEF, hMSCs, or MATRIGEL matrix (see Example 1). Following expansion on hMSCs for 6 passages, hES cell aliquots ( 1/20 or 5%) were seeded in 6-well plates containing irradiated pMEFs (n=3), hMSCs from donor #1 (n=2), hMSCs from donor #2 (n=3), or coated with MATRIGEL matrix (n=3). After six days in culture, numbers of live hES cell colonies (≧50 cells) were counted in each well. The mean and standard error of each sample is plotted. Ordinate is number of human embryonic stem cell (ESC) colonies.

### DETAILED DESCRIPTION OF THE INVENTION

The present invention undifferentiated pluripotential human embryonic stem (hES) cells that are dependent on adult human feeder cells, or an hES cell-maintaining product of said adult human feeder cells, for maintenance in culture. Embryonic stem (ES) cells are continuous proliferating pluripotential stem cell lines of embryonic origin that were first isolated from the inner cell mass (ICM) of mouse blastocysts 20 years ago. Distinguishing features of ES cells, as compared to the committed “multipotential” stem cells present in adults, include the capacity of ES cells to maintain an undifferentiated state indefinitely in culture, and the potential that ES cells have to develop into every different cell type in the body. Based on methods developed for mouse ES cells, human ES (hES) cell lines were established (Thomson et al., Science 282:1145-1147, 1998, which is incorporated herein by reference). Like mouse ES cells, hES cells can proliferate in culture for years and maintain a normal karyotype. Both mouse and human ES cells express high levels of a membrane alkaline phosphatase (APase) and of Oct-4, a transcription factor that is critical to ICM and germline formation (see, e.g., Reubinoff et al., Nature Biotechnol. 18:399-404, 2000, which is incorporated herein by reference; Thomson et al., supra, 1998). Unlike mouse ES cells, hES cells do not express the stage-specific embryonic antigen SSEA-1, but express SSEA-4, which is another glycolipid cell surface antigen recognized by a specific monoclonal antibody (see, e.g., Amit et al., Devel. Biol. 227:271-278, 2000, which is incorporated herein by reference; Thomson et al., supra, 1998).

The growth requirements of hES cells are different from those used for mouse ES cells. Prolonged propagation of hES cells generally has been achieved by co-culturing the hES cells with primary mouse embryonic fibroblasts (pMEFs), which serve as feeder cells. The existing hES cell lines were unable to maintain an undifferentiated state in the absence of supporting feeder layer cells, even when exogenous cytokines such as LIF, and gelatin-coated plates were used (see, e.g., Odorico et al., Stem Cells 19:193-204, 2001, which is incorporated herein by reference; see, also, Thomson et al., supra, 1998; Amit et al., supra, 2000). Differentiated hES cell colonies that formed either in the absence of feeder cells or after extended culture without appropriate splitting gradually lost SSA-4 and Oct-4 expression (Henderson et al., Stem Cells 20:329-37, 2002; Schuldiner et al., Proc. Natl. Acad. Sci. USA 97:11307-12, 2000, each of which is incorporated herein by reference; see, also, Reubinoff et al., supra, 2000). However, viable pMEFs may not be necessary to support hES cells, which also may be maintained on extracellular matrix (ECM) if the conditioned medium from pMEFs is provided (Xu et al., Nature Biotechnol. 19:971-974, 2001, which is incorporated herein by reference); this study used MATRIGEL matrix, which is a crude extract of basement membrane matrices from mouse sarcomas (Becton Dickson Labware; Bedford Mass.). In the latter study, however, it was unclear whether the feeder-free culture method using MATRIGEL matrix actually expanded the hES cells (i.e., net increase) or whether the undifferentiated hES cells merely were maintained in culture for the reported culture period. Nonetheless, a consequence of the use of uncharacterized rodent cells such as pMEFs, or products derived therefrom (e.g., conditioned medium), or of rodent tumor crude extracts is that xenogenic biologics can remain associated with the hES cells, thus imposing an extra risk to the clinical utility of hES cell lines (see, e.g., Odorico et al., supra, 2001).

Expanding hES cells efficiently under a clinically applicable culture condition is a prerequisite for their use in cell and gene therapies and drug discovery methods. As disclosed herein, hES cells can be expanded using human cells derived from adult BM. An improved method to expand hMSCs was developed, and the hMSCs were used to support prolonged growth of hES cells. Irradiated hMSCs from various donors at p2 to p5 supported the hES cell expansion in a serum-free medium at a rate similar to that observed using pMEFs. The hES cells expanded by co-culture with hMSCs displayed the unique morphology and molecular markers characteristic of undifferentiated hES cells, and retained a normal chromosomal karyotype.

The availability of culture-expanded and highly homogenous hMSCs allows detailed analyses to be performed that could not previously be performed using heterogeneous cell populations such as (p3) MEFs. A number of reports examined the production of cell adhesion molecules and growth factors/cytokines by hMSCs, and both mRNA and protein for LIE as well as IL-6 and IL-11 have been identified (Cheng et al., J. Cell. Physiol 184:58, 2000; Mbalaviele et al., Endocrinology 140:3736-3743, 1999). Indeed, hMSCs fully supported the proliferation of undifferentiated mouse ES cells in the absence of exogenous LIF, in either FBS-containing medium or hES cell culture medium (Cheng et al., Stem Cells 21:131-142, 2003, which is incorporated herein by reference).

Fetal skin and muscle cells from 14-week-aborted fetuses also can support prolonged growth of hES cells (Richards et al., Nature Biotech. 19:971-974, 2002). However, ethical concerns regarding the derivation of fetal cells from aborted human fetuses limit their use. Human feeder cells derived from adult fallopian tube (AFT) tissues obtained following hysterectomy also have been used to support hES cell growth. The use of primary AFT cells for culturing and expanding hES cells will not be practical, however, unless the AFT cells can be immortalized or otherwise become readily and conveniently available. In comparison, hMSCs readily can be derived from adult healthy donors or perspective patients, and can be expanded at least about one million-fold, thus providing a readily available source for use in co-cultures to support hES cell expansion (see, also, Example 2).

Several newborn foreskin fibroblast preparations have been reported to support hES cell growth (Amit et al., Biol. Reprod. “Papers in Press”, Jan. 22, 2003; see, also, Biol. Reprod. 68:2150-2156, 2003; Hovatta et al, Human Reprod. 18:1404-1409, 2003), although others reported contrasting results (Richards et al., Stem Cells 21:546-556, 2003). As disclosed herein, certain types of adult human cells, including hMSCs and CCD-1087ck breast skin fibroblasts, but not Hs27 or BJ fibroblasts, can support the growth of undifferentiated pluripotential hES cells in culture. Accordingly, the present invention provides a panel of supportive and non-supportive postnatal human fibroblast cell types that provide a means to elucidated the nature of biomolecules uniquely produced by the supportive feeder cells, and further provides isolated undifferentiated pluripotential human embryonic stem (hES) cells, wherein the hES cells exhibit dependence on adult human feeder cells, or an hES cell-maintaining product of said adult human feeder cells, for maintenance in culture.

As used herein, the term “undifferentiated pluripotential hES cells” or “hES cells” refers to human precursor cells that have the ability to form any adult cell, except placental cells. Human ES cells are derived from fertilized embryos that are less than one week old. Reference also is made herein to “multipotential stem cells”, which are cells that are destined to become a particular type of cells (e.g., hematopoietic stem cells are multipotential stem cells that are destined to differentiate into red blood cells or white blood cells), and to “terminally differentiated cells”, which are adult cells that generally perform a specific function (e.g., muscle cells, retinal cells, and neurons).

Supportive adult human feeder cells are exemplified by culture-expanded human bone marrow stromal cells (hMSCs) of passage 2 (p2) to p5, including hMSCs from multiple donors, which supported the growth of the H1 hES cell line under a serum-free condition (Example 1). Human ES cell colonies cultured on irradiated hMSC feeders amplified greater than 100 fold during a 30 day continuous culture (5 passages), and displayed the unique morphology and molecular markers characteristic of undifferentiated hES cells as were observed when cultured on pMEFs. The hES cells expressed the transcription factor Oct-4, a membrane alkaline phosphatase and the SSEA-4, but not the SSEA-1, marker. Expanded hES cells on hMSCs retained a normal diploid karyotype after 9 passages (>60 days). Similarly, a primary human CCD-1087sk fibroblasts (“1087sk cells”; ATCC CRL-2104) s, which are derived from breast skin, supported prolonged growth of hES cells in culture (Example 2). Continuous sources of human feeder cells for hES cell culture, including immortalized hMSC and 1087sk cells, were obtained by transducing the cells with a human telomerase gene (Example 2). The transduced immortalized hMSCs grew significantly faster than normal hMSCs and exhibited a transformed phenotype and, therefore, were not used in further experiments. In comparison, the transduced immortalized 1087sk adult fibroblasts (hereinafter “HAFi cells”) retained the same growth rate as the parental 1087sk cells, and supported hES cell growth. Further, conditioned medium (CM) and the ≧30 kDa fraction of CM from the transduced immortalized HAFi cells (and from pMEFs) supported hES cells cultured on the otherwise non-supportive Hs27 cells. These results demonstrate that adult human cells, and biomolecules expressed by such cells, can support the continuous cultures of undifferentiated pluripotential hES cells. As such, compositions and methods are provided that allow for the production of large numbers of hES cells that can be used, for example, in clinical procedures without risk of non-human contaminating products.

Reference herein to culture conditions that “support hES cell growth” or “expand hES cells” or “maintain hES cells” or the like, means that the culture conditions are such that hES cells can proceed through the cell cycle, and grow and divide. As such, the hES cells of the invention are characterized, in part, in that they exhibit dependence on adult human feeder cells, or an hES cell-maintaining product of said adult human feeder cells, for maintenance in culture. In particular, the hES cells, when cultured under conditions that support hES cell growth, remain in an undifferentiated pluripotential state, as evidenced, for example, by the expression of proteins such as SSEA-4, Oct-4, APase, but not SSEA-1. It should be recognized, however, that such undifferentiated pluripotent hES cells can be manipulated, for example, by impeding their progression through the cell cycle (e.g., by freezing the cells or contacting them with a cell cycle inhibitor) or by inducing them to differentiate along a particular pathway.

As used herein, the term “adult human feeder cells” refers to cells that are obtained from a post-natal human and, when cultured with hES cells, provide an advantage to the hES cells that contributes to their maintenance in culture. The human from which the feeder cells are obtained can be a male or a female, and the adult human feeder cells can be derived from any tissue. Reference herein to “supportive” adult human feeder cells means that the feeder cells, when grown in culture, modify the cell growth medium such that the medium can support hES cell growth. Such modification of a cell growth medium produces “conditioned medium”, which can contain, for example, biomolecules that are secreted from the supportive adult human feeder cells or that are expressed on the surface of such cells and enter into the medium due, for example, to cleavage or leaching from the cell surface, and act to support hES cell growth; or can be molecules that present in the cell culture medium and that are modified by the supportive adult human feeder cells or by factors produced by the feeder cells. As such, supportive adult human feeder cells can be co-cultured with hES cells, thus supporting hES cells, or the hES cells can be cultured using conditioned medium produced by such feeder cells, or an hES cell-maintaining fraction of the conditioned medium. Supportive adult human feeder cells can, but need not, bee terminally differentiated cells, and can, but need not, be immortalized. Supportive adult human feeder cells are exemplified herein by human bone marrow stromal cells (hMSCs) and by fibroblasts derived from human breast skin.

The term “hES cell-maintaining fraction of conditioned medium” is used herein to refer to an enriched portion of conditioned medium that supports or contributes to supporting hES cell growth. An hES cell-maintaining fraction of conditioned medium is exemplified herein by a fraction of conditioned medium that was obtained using a Centricon PLUS-20 centrifugal filtration device (Millipore; Bedford Mass.). Using a filtration device having a nominal molecule weight limit of about 30 kDa, biomolecules having a molecular mass of about 30 kDa and greater were retained (see Example 2). The exemplified fraction, when added to hES cells in culture with non-supportive human feeder cells, supported hES cell growth. The term “non-supportive human feeder” is used to refer to human cells that, alone, do not support hES cells and do not modify culture medium such that conditioned medium that supports hES cell growth is produced. Preferably, the non-supportive human feeder cells are adult cells, though they also can be derived from a human embryo or zygote.

The term “biomolecule” is used herein to refer to molecules that are present in conditioned medium produced by supportive adult human feeder cells and that contribute to supporting hES cell growth. As such, the term is used broadly to refer to proteins, nucleic acids, lipids, carbohydrates, lipoproteins, and the like, that are produced by supportive adult human feeder cells, as well as molecules such as growth factors, vitamins, cofactors, and the like, that are present in a cell culture medium and modified by the feeder cells or a molecule produced by the feeder cells. For example, a protein secreted by supportive adult human feeder cells can bind a cofactor present in culture medium to produce a biomolecule that contributes to supporting hES cell growth. Such biomolecules are a component of the conditioned medium that can support hES cell growth and can be enriched using methods as disclosed herein or otherwise known in the art.

The disclosed serum-free, non-human animal cell-free culture system provides a clinically useful and ethically acceptable for culturing and expanding hES cells at a scale sufficient to allow clinical use of the hES cells (or cells derived from hES cells). An additional advantage of using hMSCs to support the hES cell growth is that autologous and unrelated (allogeneic) hMSCs have been tested in a clinical transplantation setting, and do not generate alloreactive T lymphocytes in culture and in large animals (Deans and Moseley, Expt. Hematol. 28:875-84, 2000; Koc and Lazarus, Bone Marrow Transplant. 27:235-239, 2001); in fact, hMSCs down-regulate an allo-immune response of the host to the third party graft (Koc and Lazarus, supra, 2001; Bartholomew et al., Expt. Hematol. 30:42-48, 2002). As such, the presence of hMSCs, derived from a patient or from a universal source, can help to induce immune tolerance and reduce any potential allogeneic rejection of the hES cell-derived progeny (a third party graft), when transplanted into a patient whose genotype is different from the hES cells.

Although hMSCs can proliferate in culture for a long time, their proliferation rate and differentiation potential are significantly reduced after 6 passages (>25 population doublings). Their ability to support hES cell expansion was also reduced after 6 passages. Similarly, p4-5 pMEFs had reduced activity in supporting hES cell expansion as compared to p3 MEFs, which were used routinely. As such, p2-5 hMSCs can be used as disclosed herein to achieve hES cell expansion. However, hMSCs have been immortalized by over-expressing the TERT gene, the catalytic subunit of telomerase (Shi et al., Nature Biotechnol. 20:587-591, 2002; Simonsen et al., Nature Biotechnol. 20:592-596, 2002; Okamoto et al., Biochem. Biophys. Res. Comm. 295:354-361, 2002, each of which is incorporated herein by reference).

Several distinct types of multipotent stem cells have been isolated from adult BM, which is the primary site of hematopoietic stem cells, the common precursor of blood and immune cells, and also is a site for non-hematopoietic stem cells, including, for example, mesenchymal stem cells (MeSCs), which are capable of generating mesenchymal cells and stromal cells that support hematopoiesis (see, e.g., Prockop, Science 276:71-74, 1997; Bianco and Robey, J. Clin. Invest. 105:1663-1668, 2001; Bianco et al., Stem Cells 19:180-192, 2001; Dennis and Charbord, Stem Cells 20:205-214, 2002). In addition, certain freshly isolated or culture expanded BM cells can differentiate into many other types of cells, including hepatocytes in liver, neurons and glial cells in brain, satellite cells in skeletal muscles and cardiomyocytes in the heart (see, e.g., Kopen et al., Proc. Natl. Acad. Sci. USA 96:10711-10716, 1999; Woodbury et al., J. Neurosci. Res. 61:364-370, 2000; Schwartz et al., J. Clin. Invest. 109:1291-302, 2002). Thus, adult BM contains cells and a microenvironment that can maintain stem cells in a relatively undifferentiated state.

Several methods have been developed to obtain large numbers of marrow stromal progenitor cells in culture from adult human BM aspirates, either by physical enrichment of precursor cells followed by culture expansion, or by direct culture selection and amplification. These marrow fibroblastic cells have been termed “stromal progenitor cells”, reflecting their proliferation potential in culture, “marrow stromal cells” (MSCs), reflecting the source and method of the derivation, or MeSCs, reflecting their proven potentials to generate multiple types of mesenchymal cells when exposed to appropriate stimuli in vivo or in vitro (see, e.g., Pittenger et al., Science 284:143-147, 2002). Methods used to isolate MSCs and MeSC are very similar in practice and widely used (see, e.g., Cheng et al., supra, 2000). As such, marrow-derived (fibroblastic) stromal cells that are isolated by such methods and function as non-hematopoietic multipotent stem cells are referred to collectively herein as MSCs (or “hMSCs” when derived from human BM).

After two passages (approximately 14 cell divisions) in a selective medium supplemented with fetal bovine serum (FBS), culture-expanded hMSCs are morphologically and phenotypically homogenous and essentially free of endothelial cells, macrophage or adipocyte contamination (Pittenger et al., supra, 2002; Cheng et al., supra, 2000). As mentioned above, the availability of such culture-expanded and highly homogenous hMSCs has allowed detailed studies that were not possible using “mixed” stromal cell populations. For example, when used as adherent feeder cells, the culture-expanded hMSCs supported human CD34⁺ hematopoietic stem cells in long-term culture assays and their differentiation into erythroid, myeloid, megakaryocytic, osteoclastic or B cell lineages, even when cultured in the absence of added cytokines (see, e.g., Majumdar et al., J. Cell. Physiol. 176:57-66, 1998; Deans and Moseley, supra, 2000; Cheng et al., supra, 2000). The activity is due to, at least in part, the production of various hematopoietic cytokines including LIF, IL-6, IL-11 as well as SCF and Flt3/Flk2 ligand (FL) by hMSCs (Cheng et al., supra, 2000; Majumdar et al., supra, 1998; Deans and Moseley, supra, 2000).

As disclosed herein, culture-expanded hMSCs can substitute for pMEFs as feeder cells for hES cells, and fully supported prolonged expansion of undifferentiated pluripotential hES cells in culture (Example 1). For example, hES cells co-cultured on irradiated hMSCs expanded greater than 100-fold during a 30 day continuous culture (5 passages), maintained their normal karyotype after 9 passages, and retained unique hES cell morphology and expression of markers such as APase and SSEA-4, which are characteristic of undifferentiated hES cells. Furthermore, primary 1087ck adult breast skin fibroblasts, as well as 1087ck fibroblasts immortalized by transduction with a human telomerase gene, supported hES cells (Example 2). In addition, conditioned medium, and a fraction of conditioned medium containing components having a molecular weight of about 30 kDa and greater supported hES cells, particularly when the hES cells were co-cultured with non-supportive feeder cells, which, in the absence of the conditioned medium or fraction thereof, do not support hES cell growth.

Accordingly, the present invention provides a method of obtaining an expanded population of undifferentiated pluripotential hES cells by culturing hES cells with supportive adult human feeder cells, or with an hES cell-maintaining product of supportive adult feeder cells, under conditions suitable for growth of the hES cells; and further provides a culture of undifferentiated pluripotential hES cells prepared by such a method. The conditions suitable for growth of the hES cells according to a method of the invention include conditions typically used for cell culture such as those disclosed herein (see Example 1) or otherwise known in the art. For example, conditions suitable for growth of hES cells can include incubation of the hES cells at about 37° C. in an atmosphere of about 5% carbon dioxide in air and having about 95% humidity, and in a growth medium (e.g., a minimal growth medium), which can be supplemented with serum (e.g., human serum) or with a serum substitute, and with amino acids, growth factors (e.g., basic fibroblast growth factor; bFGF).

Where the method of expanding hES cells utilizes co-culture of the hES cells with supportive adult human feeder cells, the feeder cells can be any adult human cells that produce biomolecules that support the growth and proliferation of hES cells in culture. Examples of such supportive adult human feeder cells include human bone marrow stromal cells (hMSCs) and adult breast skin fibroblasts. Generally, but not necessarily, the feeder cells are irradiated with a sufficient dose of irradiation such that they remain viable, but do not proliferate. As advantage of using irradiated feeder cells is that they do not overgrow the hES cells. The supportive adult human feeder cells also can, but need not, be immortalized.

A method of expanding hES cells also can be practiced by culturing the hES cells in an hES cell-maintaining product of supportive adult feeder cells (i.e., conditioned medium or a fraction of condition medium that supports hES cell growth). According to this aspect, the hES cells can be further cultured in the presence of extracellular matrix (ECM) components, which facilitate growth of the hES cells. The ECM components can be provided as a cellular extract, or can be provided by co-culturing the hES cells with non-supportive feeder cells.

A method of obtaining an expanded population of undifferentiated pluripotential hES cells can further include a step of isolating hES cells of the expanded population, thus providing isolated undifferentiated pluripotential hES cells. Accordingly, isolated undifferentiated pluripotential hES cells obtained by such a method are provided. Methods of isolating cells such as hES cells from culture are disclosed herein or otherwise known in the art. For example, the hES cells can be removed from a tissue culture plate using collagenase, which is commonly used to collect hES cells, or trypsin, which, as disclosed herein, provided more hES cells as determined by colony formation assays. Where the hES cells are co-cultured with feeder cells, it can be desirable to further separate the hES cells from the feeder cells. Such a purification of hES cells can be performed, for example, by contacting a mixed population of hES cells and feeder (or other) cells with an antibody specific for one cell type or the other (e.g., an anti-SSEA-4 antibody, which is specific for hES cells, which express SSEA-4). Where the antibody is labeled with a fluorochrome, or is contacted with a secondary antibody so labeled, cells bound by the antibody (e.g., hES cells bound by anti-SSEA-4) can be isolated by fluorescence activated cell sorting (FACS). Where the hES cells and feeder (or other) cells have different densities or sizes, purification of hES cells can be performed, for example, by a density gradient centrifugation or centrifugal elutriation method.

An expanded population of undifferentiated pluripotential hES cells can be sub-cultured and, if desired, aliquots of cells can be reserved following one or more passages, thus providing a means to obtain large numbers of hES cells, including hES cells of different passage numbers. As such, populations of hES cells at different passages in culture are provided, as are continuous cultures of undifferentiated pluripotential hES cells. The aliquots of expanded and/or sub-cultured populations of hES cells, which can be any number of cells (e.g., about 100 cells, 1000 cells, 10,000 cells, 50,000 cells, 100,000 cells, 500,000 cells, 1,000,000 cells, or more) can be frozen and used as a source of hES cell material (e.g., an hES cell extract, hES cell nucleic acids, and hES cell proteins) or can be frozen under conditions that maintain the viability of the cells such that the frozen hES cells, upon thawing, can be used directly (e.g., in a therapeutic procedure) or expanded in culture. Methods for freezing cells in a viable condition are well known and routine in the art and, include, for example, methods using dimethylsulfoxide to prevent ice crystal formation. Accordingly, aliquots of frozen undifferentiated pluripotent hES cells obtained by a method of the invention are provided, as are pluralities of aliquots of frozen undifferentiated pluripotent hES cells, wherein, for example, two or more aliquots of a plurality contain hES cells of different passage numbers.

As disclosed herein, conditioned medium obtained by culturing supportive adult human feeder cells can support hES cell growth under conditions that would not otherwise support such growth. For example, hES cells were unable to grow when co-cultured with non-supportive Hs27 cells, but were able to grow when conditioned medium from supportive adult human feeder cells was added. Accordingly, the present invention provides a method of obtaining biomolecules that are required for growth of undifferentiated pluripotential hES cells in culture. In one aspect, such a method can be performed by culturing adult human cells that can support the growth of hES cells in culture; and isolating conditioned medium generated by culturing the adult human cells, wherein the condition medium contains biomolecules that support hES cell growth in culture. As such, the invention provides conditioned medium that supports the growth of hES cells in culture.

In another aspect, the method can further include obtaining from the conditioned medium an enriched fraction containing biomolecules that support hES cell growth in culture. As disclosed herein, an enriched fraction of conditioned medium comprising that component having a molecular mass greater than about 30 kDa supported the growth of undifferentiated pluripotent hES cells in culture. As such, the invention also provides an enriched fraction of conditioned medium that contains biomolecules that have a molecular mass greater than about 30 kDa and support undifferentiated pluripotent hES cell growth in culture. Such biomolecules, or an enriched fraction of conditioned medium containing such biomolecules, can be obtained using methods as disclosed herein or otherwise known in the art. For example, a column chromatography method can be used to collect fractions of conditioned medium, which, alone or in combination, can be examined for the ability to support hES cell growth. Further enrichment of active fractions can be effected using routine biochemical methods including, for example, salt fractionation methods, gel chromatography methods, any of various high performance liquid chromatography methods, capillary gel electrophoresis, and isoelectric focusing. As the relevant biomolecules are enriched to greater degrees, purification can be monitored, for example, by polyacrylamide gel electrophoresis.

Biomolecules that are produced by supportive adult human feeder cells also can be identified using methods of differential screening, for example, by comparing gene expression of supportive feeder cells with gene expression of non-supportive feeder cells, and identifying differentially expressed genes. By further examining the genes that are differentially expressed, biomolecules that are produced by the supportive adult human feeder cells and support hES cell growth, or that are differentially expressed in the non-supportive feeder cells and reduce or inhibit hES cell growth can be identified, thus providing a means to formulate defined media for growing hES cells in culture.

The present invention also relates to a method for obtaining undifferentiated pluripotential hES cells. Such a method can be performed, for example, by culturing a suspension of cells that includes undifferentiated pluripotential hES cells, and supportive adult human feeder cells (or an hES cell-maintaining product of said feeder cells), under conditions suitable for growth of the hES cells; and isolating cells that express SSEA-4, Oct-4, and alkaline phosphatase, and do not express SSEA-1. Accordingly, isolated undifferentiated pluripotential hES cells obtained by such a method also are provided. The suspension of cells comprising undifferentiated pluripotential hES cells can be a suspension of cells such as those available from a National Stem Cell Center, or can be a cell suspension prepared from an embryo that is no more than about one week old, for example, an embryo obtained by in vitro fertilization. In one embodiment, the method for obtaining undifferentiated pluripotential hES cells is performed by culturing the suspension comprising hES cells and the supportive adult human feeder cells (e.g., human bone marrow stromal cells). In another embodiment, the method is performed by culturing the suspension comprising hES cells, an hES cell-maintaining product of the supportive adult human feeder cells (e.g., conditioned medium), and, optionally, non-supportive feeder cells (i.e., cells that, alone, cannot support hES cell growth). Preferably, the non-supportive feeder cells are human cells (e.g., adult human cells).

A method of culturing undifferentiated pluripotential hES cells as disclosed herein can further include a step of inducing differentiation of the hES cells. Differentiation of the hES cells can induced while the cells are in culture, or the hES cells can be isolated from the culture and induced to differentiate, thus providing a means to obtain a substantially pure population of differentiated cells. Accordingly, the invention provides a population of differentiated cells obtained by such a method. As used herein, the term “substantially pure”, when used in reference to hES cells or cells derived therefrom (e.g., differentiated cells), means that the specified cells (e.g., differentiated cells) constitute the majority of cells in the preparation (i.e., more than 50%). Generally, a substantially purified population of cells constitutes at least about 70% of the cells in a preparation, usually about 80% of the cells in a preparation, and particularly at least about 90% of the cells in a preparation (e.g., 95%, 97%, 99% or 100%). As such, a method of the invention provides the advantage that a substantially pure population of a particular type of cells (e.g., hES cells induced to differentiate into neurons) can be obtained without contamination by other cell types. Such substantially pure populations of differentiated cells can be multipotential cells or terminally differentiated cells, and can be used, for example, in screening assays or for therapeutic purposes.

The present invention also relates to a method for identifying an agent that alters a function of an undifferentiated pluripotential hES cell by contacting hES cells with a test agent, wherein the hES cells exhibit dependence on adult human feeder cells, or an hES cell-maintaining product of said adult human feeder cells, for maintenance in culture; and detecting a change in a function of the hES cells in presence of the test agent as compared to the function in the absence of the test agent, thereby identifying the test agent as an agent that alters the function of the hES cells. The term “test agent” is used broadly herein to mean any molecule that is being examined for an ability to alter a function of an hES cell according to a method of the invention. Although the method generally is used as a screening assay to identify previously unknown molecules that have a desired activity, e.g., that act as agonists or antagonists of molecules that are known to alter an hES cell function, the methods of the invention also can be used to confirm that an agent known to have a particular activity in fact has the activity, for example, in standardizing the activity of the agent.

A test agent examined according to a method of the invention can be any type of molecule, for example, a polynucleotide, a peptide, a peptidomimetic, peptoids such as vinylogous peptoids, a small organic molecule, or the like, and can act in any of various ways to alter a function of an hES cell. For example, the test agent can act extracellularly by binding to a cell surface receptor expressed by hES cells, thereby altering a function mediated by binding of a ligand that generally binds to and acts via the receptor. Alternatively, the test agent can be one that traverses the hES cell membrane, either passively or via an active transport mechanism, and acts within an hES cell to alter a function.

A peptide test agent can be any polymer of amino acids or amino acid analogs, and can vary from about three to four residues to hundreds or thousands. As such, it should be recognized that the term “peptide” is not used herein to suggest a particular size or number of amino acids comprising the molecule, and that a peptide test agent can contain up to several amino acid residues or more. Peptide test agents can be prepared, for example, by a method of chemical synthesis, or using methods of protein purification, followed by proteolysis and, if desired, further purification by chromatographic or electrophoretic methods, or can be expressed from an encoding polynucleotide. Further, a peptide test agent can be based on a known peptide, for example, a naturally occurring peptide, but can vary from the naturally occurring sequence, for example, by containing one or more D-amino acids in place of a corresponding L-amino acid; or by containing one or more amino acid analogs, for example, an amino acid that has been derivatized or otherwise modified at its reactive side chain. Similarly, one or more peptide bonds in the peptide test agent can be modified, or a reactive group at the amino terminus or the carboxy terminus or both can be modified. Such peptides can have improved stability to a protease, an oxidizing agent or other reactive material the peptide test agent may encounter in a biological environment. Such peptide test agents also can be modified to have decreased stability in a biological environment such that the period of time the peptide is active in the environment is reduced.

A polynucleotide test agent also can be examined according to a method of the invention. The term “polynucleotide” is used herein to mean a sequence of two or more deoxyribonucleotides or ribonucleotides that are linked together by a phosphodiester bond. As such, the term “polynucleotide” includes RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the term “polynucleotide” as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR). In various embodiments, a polynucleotide of the invention can contain nucleoside or nucleotide analogs, or a backbone bond other than a phosphodiester bond.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234, 1994; Jellinek et al., Biochemistry 34:11363-11372, 1995; Pagratis et al., Nature Biotechnol. 15:68-73, 1997, each of which is incorporated herein by reference).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986, 1994; Ecker and Crooke, BioTechnology 13:351360, 1995, each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995).

A polynucleotide test agent can be contacted with or introduced into an hES cell using methods as disclosed herein or otherwise known in the art. Generally, but not necessarily, the polynucleotide is introduced into the cell, where it effects its function either directly, or following transcription or translation or both. For example, as mentioned above, the polynucleotide can encode a peptide test agent, which is expressed in the hES cell and alters a function of the cell. A polynucleotide test agent also can be, or can encode, an antisense molecule, a ribozyme or a triplexing agent, which can be designed to target one or more specific target nucleic acid molecules.

Antisense polynucleotides, ribozymes and triplexing agents generally are designed to be complementary to a target sequence, which can be a DNA or RNA sequence, for example, mRNA, and can be a coding sequence, a nucleotide sequence comprising an intron-exon junction, a regulatory sequence such as a Shine-Delgarno sequence, or the like. The degree of complementarity is such that the polynucleotide, for example, an antisense polynucleotide, can interact specifically with the target sequence in a cell. Depending on the total length of the antisense or other polynucleotide, one or a few mismatches with respect to the target sequence can be tolerated without losing the specificity of the polynucleotide for its target sequence. Thus, few if any mismatches would be tolerated in an antisense molecule consisting, for example, of 20 nucleotides, whereas several mismatches will not affect the hybridization efficiency of an antisense molecule that is complementary, for example, to the full length of a target mRNA encoding a cellular polypeptide. The number of mismatches that can be tolerated can be estimated, for example, using well known formulas for determining hybridization kinetics (see Sambrook et al., supra, 1989) or can be determined empirically using methods as disclosed herein or otherwise known in the art, particularly by determining that the presence of the antisense polynucleotide, ribozyme, or triplexing agent in a cell decreases the level of the target sequence or the expression of a polypeptide encoded by the target sequence in the cell.

A polynucleotide useful as an antisense molecule, a ribozyme or a triplexing agent can inhibit translation or cleave the nucleic acid molecule, thereby altering a function of an hES cell. An antisense molecule, for example, can bind to an mRNA to form a double stranded molecule that cannot be translated in a cell. Antisense oligonucleotides of at least about 15 to 25 nucleotides are preferred since they are easily synthesized and can hybridize specifically with a target sequence, although longer antisense molecules can be expressed from a polynucleotide introduced into the target cell. Specific nucleotide sequences useful as antisense molecules can be identified using well known methods, for example, gene walling methods (see, for example, Seimiya et al., J. Biol. Chem. 272:4631-4636, 1997, which is incorporated herein by reference). Where the antisense molecule is contacted directly with a target cell, it can be operatively associated with a chemically reactive group such as iron-linked EDTA, which cleaves a target RNA at the site of hybridization. A triplexing agent, in comparison, can stall transcription (Maher et al., Antisense Res. Devel. 1:227, 1991; Helene, Anticancer Drug Design 6:569, 1991).

A screening assay of the invention can be performed by contacting the test agent and hES cells in vivo, for example, following administration or implantation of the hES cells into a subject, or by contacting the test agent an hES cells in vitro, for example, by adding the test agent to a culture containing the hES cells or to hES cells isolated from a culture. The function of undifferentiated pluripotential hES cell that can be altered due to contact with an agent can be any function of the hES cells. For example, the function can be expression of gene that typically is expressed (or not expressed) in hES cells, and the agent can alter the function by increasing or decreasing the level of expression of an expressed gene (e.g., decreasing expression of stage-specific surface antigen-4, alkaline phosphatase, or Oct-4 transcription factor), or by turning on the expression of an unexpressed gene (e.g., inducing expression of stage-specific surface antigen-1), in the hES cells. In one embodiment, the agent that effects a function of hES cells is one that induces differentiation of the hES cells, thereby producing differentiated cells. Such differentiated cells can be multipotential human stem cells (e.g., hematopoietic stem cells) or can be terminally differentiated cells (e.g., muscle cells, neuronal cells, blood cells, connective tissue, or epithelial cells). As such, the method can be used to identify an agent that induces differentiation of hES cell to pancreatic beta cells, hepatocytes, cardiomyocytes, skeletal muscle cells, or any other cell type.

A screening method of the invention provides the advantage that it can be adapted to high throughput analysis and, therefore, can be used to screen combinatorial libraries of test agents in order to identify those agents that can alter a function of an hES cell. Methods for preparing a combinatorial library of molecules that can be tested for a desired activity are well known in the art and include, for example, methods of making a phage display library of peptides, which can be constrained peptides (see, for example, U.S. Pat. No. 5,622,699; U.S. Pat. No. 5,206,347; Scott and Smith, Science_{—}249:386-390, 1992; Markland et al., Gene 109:13-19, 1991; each of which is incorporated herein by reference); a peptide library (U.S. Pat. No. 5,264,563, which is incorporated herein by reference); a peptidomimetic library (Blondelle et al., Trends Anal. Chem. 14:83-92, 1995; a nucleic acid library (O'Connell et al., et al., Proc. Natl. Acad. Sci., USA 93:5883-5887, 1996; Tuerk and Gold, Science 249:505-510, 1990; Gold et al., Ann. Rev. Biochem. 64:763-797, 1995; each of which is incorporated herein by reference); an oligosaccharide library (York et al., Carb. Res. 285:99-128, 1996; Liang et al., Science 274:1520-1522, 1996; Ding et al., Adv. Expt. Med. Biol. 376:261-269, 1995; each of which is incorporated herein by reference); a lipoprotein library (de Kruif et al., FEBS Lett. 399:232-236, 1996, which is incorporated herein by reference); a glycoprotein or glycolipid library (Karaoglu et al., J. Cell Biol. 130:567-577, 1995, which is incorporated herein by reference); or a chemical library containing, for example, drugs or other pharmaceutical agents (Gordon et al., J. Med. Chem. 37:1385-1401, 1994; Ecker and Crooke, BioTechnology 13:351-360, 1995; each of which is incorporated herein by reference). Polynucleotides can be particularly useful as agents that can alter a function of hES cells because nucleic acid molecules having binding specificity for cellular targets, including cellular polypeptides, exist naturally, and because synthetic molecules having such specificity can be readily prepared and identified (see, for example, U.S. Pat. No. 5,750,342, which is incorporated herein by reference).

For a high throughput format, hES cells can be introduced into wells of a multiwell plate or of a glass slide or microchip, and can be contacted with the test agent. Generally, the cells are organized in an array, particularly an addressable array, such that robotics conveniently can be used for manipulating the cells and solutions and for monitoring the hES cells, particularly with respect to the function being examined. An advantage of using a high throughput format is that a number of test agents can be examined in parallel, and, if desired, control reactions also can be run under identical conditions as the test conditions. As such, the methods of the invention provide a means to screen one, a few, or a large number of test agents in order to identify an agent that can alter a function of hES cells, for example, an agent that induces the hES cells to differentiate into a desired cell type, or that prevents spontaneous differentiation, for example, by maintaining a high level of expression of regulatory molecules such as Oct-4.

The present invention also provides a method of ameliorating a pathologic condition in a subject by administering undifferentiated pluripotential hES cells, which are cultured as disclosed herein, or cells derived from said hES cells, to the subject, wherein the hES cells exhibit dependence on adult human feeder cells, or an hES cell-maintaining product of said adult human feeder cells, for maintenance in culture. As used herein, the term “ameliorate” means that signs or symptoms associated with the condition are lessened. Methods of determining whether signs or symptoms of a particular condition are ameliorated will depend on the particular condition, and will be well known to the skilled clinician. For example, where the condition being treated is a spinal cord injury, the skilled clinician will know that a therapeutic benefit can be identified by detecting voltage transmission in neurons known to have been affected by the injury, thus indicating amelioration of the condition.

It is well recognized that hES cell therapy holds the promise for treating a wide variety of diseases, including hereditary disorders (e.g., Parkinson's disease) and disorders associated with aging (e.g., glaucoma), as well as providing a means to treat injuries that cannot presently be treated (e.g., spinal cord injuries). As such, the pathologic condition to be treated according to a method of the invention can be any condition amenable to treatment using hES cells cultured as disclosed herein, or differentiated cells derived from such hES cells. Examples of such pathologic conditions include degenerative disorders (e.g., neurodegenerative disorders such as Alzheimer's disease, multiple sclerosis (MS), Parkinson's disease, muscular dystrophy, amyotrophic lateral sclerosis, and autism); ocular disorders such as glaucoma, retinitis pigmentosa, and macular degeneration; autoimmune disorders such as systemic lupus erythematosis, rheumatoid arthritis, diabetes, and MS; viral conditions such as hepatitis C infection and acquired immune deficiency disorder; heart and circulatory conditions such as myocardial infarction and atherosclerosis; adrenal disorders such as Addison's disease; kidney disease, liver disease, lung disease or other such disorder that can require an organ transplant; or a condition result from an injury such as, for example, a spinal cord a burn or a stroke; conditions associated with aging (e.g., hair loss and weight control); organ or tissue cancers such as blood cancers (or in combination with a therapy such as chemotherapy to replace cells damaged by the therapy); other blood disorders such as Wiscott Aldrich syndrome; or any other condition in which hES cells can be used to restore, regenerate, or otherwise ameliorate signs and/or symptoms associated with the disorder.

The following examples are intended to illustrate but not limit the invention.

### EXAMPLE 1

### Human Embryonic Stem Cell Growth in Co-Cultures with Adult Human Bone Marrow Stromal Cells

This example demonstrates that human bone marrow stromal cells (hMSCs) fully support the growth of undifferentiated pluripotent hES cells in continuous cultures.

Isolation and Expansion of hMSCs:

Bone marrow (BM) samples collected from healthy and consented human donors were purchased from AllCells company (Berkeley, Calif.), or can be collected using routine clinical methods. Mononuclear cells (MNCs) were isolated from heparinized BM aspirates (diluted with equal volume of phosphate buffered saline) by the standard density (1.077 g/ml) centrifugation using FICOLL density gradient medium (Pharmacia; Piscataway N.J.). As compared to a previous protocol (Pittenger et al., supra, 2002; Cheng et al., supra, 2000) using PERCOLL density gradient medium (1.073 g/ml; Pharmacia), the FICOLL medium method yielded 2-fold more MNCs, but generated the same total numbers of MSCs after culture expansion, per unit volume of BM samples. MNCs at the interface were recovered, washed and resuspended in hMSC medium (Dulbecco's Modified Eagles Medium (DMEM) with low glucose (Invitrogen Corp.; Carlsbad Calif.), 10% fetal bovine serum (FBS), 1% antibiotic-anti-mycotic stock solution (Invitrogen Corp.); see Pittenger et al., supra, 2002; Cheng et al., supra, 2000), in the absence or presence of 1 ng/ml basic fibroblast growth factor (bFGF; Invitrogen Corp.). The addition of bFGF to the hMSC medium resulted in consistent and optimal growth with different FBS batches from various suppliers (Hyclone Laboratories; Logan Utah; Invitrogen Corp.; Gemini; Calabasas Calif.).

For primary hMSC cultures, MNC cells were plated into 175 cm² flasks at a density of 6×10⁷ cells/flask and the cultures were incubated at 37° C. in 5% CO, in air and 95% humidity. The medium was exchanged after 48 hours, and every 3-4 days thereafter. When cells in the primary passage reached approximately 90% confluence (approximately 2 weeks), hMSCs were recovered by treatment with 0.05% trypsin/0.53 mM EDTA solution (Invitrogen Corp.) and replated into passage culture at a density of 5,000 to 10,000 cells per cm². When the cells were again confluent (10-14 days), they were harvested (passage 1; p1), then seeded, as above, to obtain passage 2 (p2) cells, and so on.

Human ES Cell Culture:

The H1 hES cell line (passage 22—“p22”) was obtained from the WiCell Research Institute (Wisconsin Wis.) and, initially, cultured as instructed by the provider (note: H1 hES cells are referred to as “WA01” in the NIH Embryonic Stem Cell Registry). Primary MEFs (p3; purchased from Specialty Media, Inc.; Phillipsburg N.J.—see “www”, at URL “specialtymedia.com”) were used, initially, as feeder cells for the hES cells.

Following irradiation with 50 Gray using a ¹³⁷Cs gamma-irradiator, approximately 200,000 pMEFs or hMSCs were plated per 9.4 cm² well in 6-well plates. The hES cell culture medium was 80% (v/v) KNOCKOUT- (KO-) DMEM, 20% (v/v) of KO Serum Replacement, 2 mM glutamine, 10 mM non-essential amino acids, 50 μM β-mercaptoethanol and 4 ng/ml bFGF (Invitrogen Corp.). Cell cultures were incubated at 37° C. in 5% CO, in air and 95% humidity. When hES cell colonies grew to a maximal size, and before the onset of visible differentiation, cells in the co-culture (hES cells and irradiated pMEFs) were digested and seeded onto freshly-prepared feeder cells. Initially, collagenase IV (1 mg/ml) was used to split cells (1:1 to 1:3), as instructed (Thomson et al, supra, 1998; Amit et al., supra, 2000). In later experiments, cells were harvested by treating the co-cultures with 0.05% trypsin/0.53 mM EDTA solution for 5 min; trypsin then was inactivated by adding soybean trypsin inhibitor (Sigma; St. Louis Mo.). The cells then were washed, and the dissociated cells in the hES cell culture medium were split from 1:1 to 1:50, and seeded onto feeder cells or 6-well plates coated with diluted (1/20) MATRIGEL matrix (Becton Dickson Labware; see, Xu et al., supra, 2001).

Immunofluorescence and APase Staining:

Co-cultures used for APase staining or immuno-fluorescence analysis were established in either 6-well or 24-well plates. Prior to analysis, adherent cell layers were fixed by the addition of 10% formalin (15 min). After washing with a Tris-based saline solution, APase staining was performed using a kit containing BCIPMBT as the substrate (Sigma). The dark blue staining was visualized by light microscopy. The fixed cells in co-cultures also were stained with mouse monoclonal antibodies (mAbs) specific for SSEA-4 (clone MC-8 13-70, isotype IgG3) or SSEA-1 (clone MC-480, isotype IgM); hybridoma supernatants of both mAbs were obtained from Developmental Studies Hybridoma Bank (Iowa City Iowa).

For immunofluorescence staining, the fixed cells were incubated 15 minutes with goat serum (2%) to block non-specific binding, then the co-cultures were stained with diluted (1:100) hybridoma supernatants specific for SSEA-4 or for SSEA-1 antigen. After incubation in the dark for 1 hr at 25° C., or overnight at 4° C., fixed cells were washed extensively, then the secondary staining reagent was added. Goat anti-mouse IgG conjugated to the ALEXA 546 fluorochrome (Molecular Probes; Portland Oreg.) was added for 45 min at 25° C. The nuclei of hES cells and hMSCs were counter-stained by Hoechst 33358 stain (Molecular Probes). Immunofluorescence analysis was performed with a Nikon (TE300) microscope with separate filters for either Hoechst 33358 stain (blue) or ALEXA 546 fluorochrome (red), or with a triple filter for blue, green and red fluorescence, simultaneously. The fluorescence and light images were recorded using Kodak® film (ASA400). The scanned image was analyzed using Adobe PHOTOSHOP 4.0 software.

Cell Isolation by Magnetic Cell Sorting (MACS):

Cells were harvested from co-cultures by gentle digestion with 0.25% trypsin/0.53 mM EDTA solution and washed once with PBS containing 2% BSA and 2 mM EDTA. Before incubating with the SSEA-4 mAb (1:100), cells were pre-incubated with human IgG (2 mg/ml) to block non-specific IgG binding. The SSEA-4 labeled cells were incubated with magnetic beads conjugated with anti-mouse IgG antibodies (Miltenyi Biotec; Auburn Calif.). The labeled cells were isolated using the miniMACS™ magnet stand and the large cell isolation column, as instructed (Miltenyi Biotec).

Flow Cytometric Analysis:

Cells were harvested as described above and suspended in 1001 μl staining buffer (2% BSA, 2 mM EDTA and 0.1% sodium azide in PBS) containing human IgG to block non-specific IgG binding. Diluted (1:100) SSEA-1 mAb or SSEA-4 mAb was added as a primary antibody. FITC or R-phycoerythrin (PE) conjugated anti-mouse IgG antibodies were used to detect binding of the SSEA-4 mAb (mouse IgG3), and anti-mouse IgM antibodies conjugated with PE were used to detect binding of the SSEA-1 mAb (secondary reagents were purchased from Caltag; Burlingame Calif., or Becton Dickinson/PharMingen; San Jose Calif.). In addition, PE conjugated mAbs recognizing SH-2/endoglin/CD105 (clone SN6 or 266), HLA-ABC/MHC class I (clone TU149), HLA-DR/MHC class II (clone L233), CD133 (clone AC133-1), Thy-1/CD90 (clone F15-42-1-5), CD34 (Clone BPCA-2) and PECAM-1/CD31 (MBC78.2), were used in conjunction with SSEA-4 and the FITC conjugated anti-mouse IgG antibody (for hES cells); the directly PE conjugated mAbs were purchased from Caltag, Miltenyi Biotec, Beckman Coulter (Miami Fla.) or Becton Dickinson/PharMingen, and used as instructed by providers. A FACScan® flow cytometer (Becton Dickinson) was used for these analyses. Ten thousand events were acquired for each sample and analyzed using CellQuest™ software (Becton Dickinson).

Karyotype Analyses of hES Cells:

Karyotyping of hMSCs or hES cells was carried out by the Laboratory of Prenatal and Research Cytogenetics in Department of Obstetrics and Gynecology at the Johns Hopkins Hospital before and after co-culturing hES cells on hMSCs (see Shamblott et al., Proc. Natl. Acad. Sci. USA 95:13726-31, 1998, which is incorporated herein by reference). Briefly, cells in co-culture were incubated with 0.1 μg/ml of colcemid for 3-4 hr, trypsinized, resuspended in 0.075 M KCl, incubated for 20 min at 37° C., then fixed in 3:1 methanol/acetic acid. After staining, karyotypes of normal human chromosomes were examined by cytogenetics specialists at the 300-band level of resolution.

RNA Preparation and Gene Expression Analysis:

The RNeasy™ RNA isolation kit (Qiagen, Valencia, Calif.) was used to extract total RNA from MACS-isolated hES cells that were co-cultured with either pMEFs or hMSCs, or from control pMEFs or hMSCs cultured in hES cell culture medium. Contaminating genomic DNA was eliminated by DNase I digestion. First strand cDNA synthesis was performed using SUPERSCRIPT II reverse transcriptase (RT) and oligo(dT)₁₂₋₁₈ as primers (Invitrogen Corp.). Aliquots (10%) of the RT product were used as a template for PCR amplification with specific primer sets for either human Oct-4 or human/mouse β-actin gene.

The oligonucleotide primer pairs for Oct-4 RT-PCR (Schuldiner et al., supra, 2000) were as follows:

(SEQ ID NO:1)

| | |
|---|---|
| Oct-4 sense: | 5′-CGTGMGCTGGAGAAGGAGAAGCTG-3′; |
| and | |

(SEQ ID NO:2)

| | |
|---|---|
| Oct-4 antisense: | 5′-CAAGGGCCGCAGGTTACACATGTTC-3′. |

The two primers correspond to nucleotides 862-886 and nucleotides 4527-4551, respectively, of the Oct-4 gene (GenBank Acc. No. Z11900); the target sequences are located in two different exons. The detected cDNA fragment by RT-PCR was 140 bp long as predicted.

The primers for the β-actin gene (GenBank Acc. No. BC016045) were as follows:

(SEQ ID NO:3)

| | |
|---|---|
| Actin-sense: | 5′-GCTCGTCGTCGACAACGGCTC-3′; |
| and | |

(SEQ ID NO:4)

| | |
|---|---|
| Actin-antisense: | 5′-CAAACATGATCTGGGTCATCTTCTC-3′. |

The detected RT-PCR product of human and mouse β-actin cDNA was 353 bp long, as expected. After 40 cycles of PCR with an annealing temperature at 60° C., the RT-PCR products were visualized by ethidium bromide staining, following electrophoresis through a 1.5% agarose gel.
Results

Growth of hES Cells

H1 hES cells (WiCell Research Institute) were expanded in co-culture on irradiated (mitotically inactive) pMEFs; the hES cells were continuously cultured for 3 months and split using collagenase IV approximately once a week, as instructed by the provider. Consistent with the provided protocol, the hES cells exhibited approximately a 2-fold expansion every passage. Upon obtaining a sufficient number of hES cells, an effort was made to improve culture conditions and splitting (passaging) methods. Consistently more uniform and greater number of hES cell colonies were obtained when the cells were split using trypsin/EDTA digestion, as compared to the collagenase method. Thus, the trypsin/EDTA digestion method was used to maintain and expand hES cells co-cultured on either pMEFs or hMSCs.

Isolation and Growth of hMSCs

Human MSCs were derived from adult BM and cultured as previously described (Pittenger et al., supra, 2002; Cheng et al., supra, 2000), except that the culture medium containing 10% FBS was supplemented with 1 ng/ml bFGF. Consistent with a previous report (Martin et al., Endocrinology 138:4456-62, 1997), addition of the low concentration of bFGF provided a consistently optimal growth condition and essentially alleviated the need to screen favorable FBS lots. Human MSCs were efficiently and consistently derived and expanded from multiple different male and female donors. After a primary and secondary passages in culture for a total time of less than or equal to 6 weeks, 75-200 million (p2) hMSCs were obtained from about 100×10⁶ MNCs present in a 10 cc BM aspirate sample. The expanded hMSCs were highly uniform in morphology and phenotypes; were essentially free of adipocytes, hematopoietic cells (CD45⁺) and endothelial cells (CD34⁺ or CD31⁺); and expressed unique markers such as CD105 (also known as SH-2 and endoglin) and Thy-1/CD90 (see Table 1).

TABLE 1
Expression of selected cell surface markers on culture expanded hMSCs and hES cells (on hMSCs)

| Cell | | | | Endoglin | MHC | MHC | AC133 | Thy-1 | | PECAM-1 | |
|---|---|---|---|---|---|---|---|---|---|---|---|
| Type | APase | SSEA-1 | SSEA-4 | (CD105) | class I | class II | (CD133) | (CD90) | CD34 | (CD31) | CD45 |
| hMSCs | − | − | − | ++ | ++ | − | − | ++ | − | − | − |
| hES | ++ | − | ++ | − | + | − | + | + | − | − | − |
| cells | | | | | | | | | | | |

−: no difference or <2 fold higher than background;
+: 2-10 fold above background;
++: >10 fold higher than background.

Prolonged Expansion of hES Cells Co-Cultured on hMSCs

Undifferentiated hES cell colonies formed on hMSC feeders in the serum-free hES cell culture medium, although they initially had a growth rate that was lower than that of hES cells grown on pMEFs. The hES cells were continuously cultured on irradiated or non-irradiated hMSCs for an additional 4 passages (approx. 4 weeks, with split ratios from 1:2 to 1:5 in each passage), then characterized in detail. In the absence of seeded hES cells, no hES cell-like colonies formed in irradiated or non-irradiated hMSC cultures. When cultured with non-irradiated hMSCs, hES cell colonies had a better growth rate and showed a more compact morphology; however, the proliferation of hMSCs in the co-culture imposed a practical difficulty—overgrowth of the hMSC feeder cells, which divided faster than hES cells. Accordingly, in subsequent experiments, hES cells were co-cultured exclusively with irradiated hMSCs as feeder cells. Using irradiated hMSCs, hES cell colonies amplified greater than 100-fold during 30 days of continuous culture, including 5 passages. In multiple experiments performed thus far, hES cells co-cultured with irradiated hMSCs have been through 13 passages.

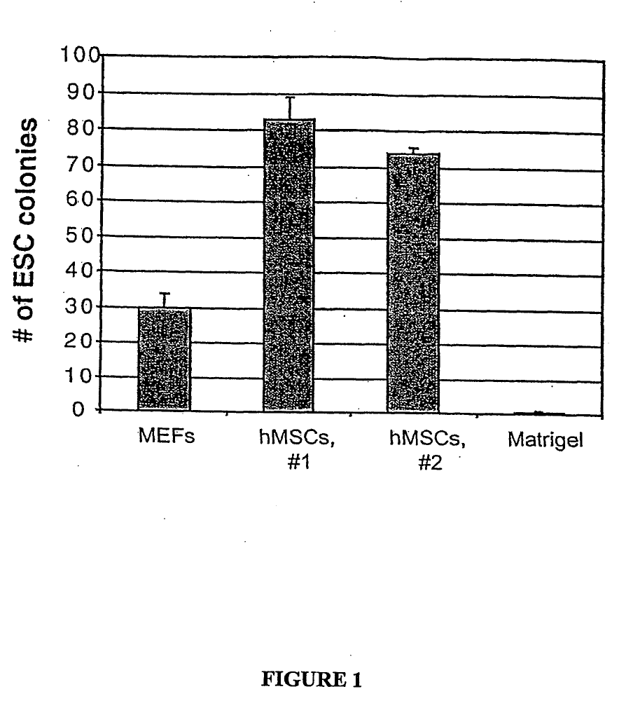

The growth of hES cells on hMSCs was compared using preparations of hMSCs obtained from various donors, and with growth on pMEFs. A 1:20 split of p6 hES cells on hMSCs (donor #1) was seeded onto duplicate wells of irradiated pMEFs or of irradiated hMSCs from donor #1 or donor #2. After six days, hES cell colonies containing ≧50 cells with an undifferentiated morphology were counted (FIG. 1). Both hMSC feeder cell populations gave rise to similar numbers of hES cell colonies; with an estimated ≧5 fold expansion in this passage. Different preparations (p2 to p5) of hMSCs from 3 donors (two males and one female) also gave similar results. Under the same culture conditions, MATRIGEL matrix did not support hES cell growth, regardless whether the conditioned medium from hMSCs or pMEFs was included in the culture. The pMEF feeder cells gave rise to fewer and smaller hES cell colonies (approximately 2-fold expansion; FIG. 1). These results indicate that hES cells grew better on hMSCs than on pMEFs once the hES cells had adapted to growth on hMSCs (6 passages in this experiment). Adaptation also may explain why the hES cells grew more poorly on hMSCs when they initially were passaged from the co-culture with MEFs.

hES Cells Co-Cultured with hMSCs Retained an Undifferentiated Phenotype:

After 4 or more passages on hMSCs, aliquots of the expanded hES cells were analyzed for the expression of cell-surface markers such as APase and SSEA-4. The APase isoform on ES cells is likely EC.3.1.3.1, which also is known as the liver/kidney/bone APase and the tissue non-specific APase (Henderson et al., supra, 2002) and is sensitive to levamisol inhibition. By histochemical staining, ES cells colonies were strongly APase positive, while hMSCs as feeder cells mostly were negative. APase activities were preferentially expressed on cell membrane of hES cells and sensitive to levamisol inhibition. Similar to that observed with irradiated pMEFs, few singular hMSCs having an apoptotic morphology (broad and flat) displayed a weak APase activity. The absence or low level of APase activities on the viable hMSCs is consistent with the report that undifferentiated hMSCs are APase negative until induced to differentiate to osteoblasts (Bruder et al., Bone 21:225-235, 1997).

The expanded hES cells also were stained for SSEA-4, a glycolipid antigen expressed on hES cells but not on mouse ES cells. Following fixation, the co-cultures of hES cells and irradiated hMSCs (p4) were stained with or without a mouse mAb against the SSEA-4 antigen. A high level of SSEA-4 expression was found in expanded hES cells, and was absent in the hMSC feeder cells.

The mAb specific for the SSEA-4 surface antigen was used in conjunction with MACS to isolate live undifferentiated hES cells that expressed SSEA-4 (SSEA-4⁺) following co-culture on hMSC or pMEF feeder cells. Cells in each of the co-cultures were isolated as a single cell suspension and pooled for each group, then the labeled SSEA-4⁺ cells were isolated using the MACS system (Miltenyi Biotec). Cells also were analyzed by flow cytometry before or after the MACS system isolation; flow cytometric analysis further confirmed that the hES cell fraction (9.6%) retained a high level of the SSEA-4 expression. Following MACS system isolation, the purity of hES cells (SSEA-4⁺) was about 95%. This result demonstrates that the disclosed method allows the isolation of hES cells that are substantially free from feeder cells.

Highly purified hES cells cultured on either hMSCs or pMEFs were analyzed for expression of the Oct-4 gene, which encodes a transcription factor that is preferentially expressed in undifferentiated pluripotential hES cells (see, e.g., Thomson et al., supra, 1998). RT-PCR analysis revealed a high level of the Oct-4 expression in hES cells cultured with pMEFs, as previously described. As disclosed herein, hES cells that were cultured with hMSCs for 5 passages also expressed the Oct-4 gene at a high level; hMSCs cultured alone had a very low but detectable level of Oct-4 gene expression.

The expression of other cell-surface markers on hES cells co-cultured with hMSCs, or on hMSCs cultured alone, also was examined by flow cytometry. The expression pattern of these unique markers (see Table 1) was consistent with previously reports for H1 and other hES cell lines (Kaufman et al., Proc. Natl. Acad. Sci. USA 98:10716-10721, 2001; Drukker et al., Proc. Natl. Acad. Sci. USA 99:9864-9869, 2002; Henderson et al., supra, 2002). The morphology, Oct-4 gene RT-PCR analysis, and expression of 11 unique cell-surface markers including APase and SSEA-4 demonstrate that hES cells expanded by co-culture with hMSCs retained the unique morphology and phenotype characteristic of undifferentiated pluripotential hES cells as previously described for hES grown on pMEFs.

hES Cells Expanded on hMSCs Have a Normal Karyotype

The H1 ES cell line, which was derived from a male embryo, retained a normal 44+XY karyotype following continuous expansion on pMEFs for 3 months. Chromosomal karyotype also was examined in one continuous co-culture (9 passages) of hES on irradiated hMSCs. To easily identify hES cells and distinguish them from hMSCs that also were present in the co-cultures, the number of human feeder cells, particularly those of the male hMSCs that were used in the first 7 passages, was reduced by seeding the hES cells onto female hMSC feeder cell for two more passages, followed by two additional passages on pMEFs. Karyotyping revealed that all 5 samples examined displayed the same normal 44+XY chromosomal karyotype as was found in the original H1 cells. These results demonstrate that the hES cells retained a stable and normal karyotype after prolonged expansion on hMSCs.

### EXAMPLE 2

### Adult Human Cells and Biomolecules Produced by the Cells Support Human ES Cell Growth

This example confirms that adult cells can support hES cell growth, and further identifies the presence of factors in conditioned medium that support hES cell growth.

In order to determine whether adult cells other than hMSCs could support hES cell growth, and to elucidate secreted factors required by hES cells, a variety of postnatal human fibroblast cell lines were examined as feeder cells. In addition to hMSCs, a primary human fibroblast cell line derived from breast skin, CCD-1087sk cells (ATCC CRL-2104) supported prolonged growth of hES cells in culture. In comparison, ATCC Hs27 fibroblasts (ATCC CRL-1634) and BJ fibroblasts (ATCC CRL-25422), which are derived from foreskin, and WI-38 fibroblasts, which are derived from fetal lung, did not support hES cell growth. These results demonstrate that hMSCs are not the only adult human cells that can be used as feeder cells to support hES cell growth.

In order to obtained immortalized human cells useful as feeder cells for hES cell growth, hMSC and 1087sk cells were transduced with a human telomerase gene and examined for hES cell growth activity (Shi et al., supra, 2002; Simonsen et al., supra, 2002; Okamoto et al., supra, 2002). Briefly, cells were plated in MEM containing sodium bicarbonate, pyruvate, non-essential amino acids, and 10% FBS, and transduced using the pBabe-hTERT-hygro vector after p9. Cells were selected with 50 μg/ml hygromycin B for at least two passages, then passaged weekly in parallel with untransduced cells; cell proliferation rate was monitored starting with p13 after transduction. Because the transduced immortalized hMSCs grew significantly faster than normal hMSCs and exhibited a transformed phenotype, they were not examined further. In comparison, the transduced immortalized 1087sk cells (hereinafter “HAFi cells”) retained the same growth rate as the parental 1087sk cells, and supported hES cell growth as determined by APase expression. The transduced HAFi cells maintained a normal karyotype (44+XX) at p30 following transduction (14 of 14 cells examined), and at p41 continue to support hES growth.

In order to determine whether biomolecules produced by the hMSC and by the immortalized HAFi cells supported hES cell growth, conditioned medium was collected following incubation of the hMSC or HAFi cells in hES cell medium. Human ES cells were plated with non-supportive Hs27 cells and conditioned medium was added. As above, undifferentiated hES cells did not grow when cultured with the Hs27 cells, alone. However, the addition of conditioned medium from the HMSC cells or HAFi cells to co-cultures of hES cells and Hs27 cells resulted in growth of undifferentiated pluripotential hES cells, as confirmed by measuring APase expression. These results demonstrate that hMSC and HAFI cells produce biomolecules that are present in conditioned medium obtained by culturing the cells.

The conditioned medium collected from hMSCs and from HAFi human feeder cells, as well as from pMEFs, were separated into two fractions using a Centricon PLUS-20 centrifugal filtration device. The initial filter used in the device had a nominal molecule weight limit (NMWL) of 5 kDa, thus retaining all molecules with a molecular weight greater than about 5 kDa. For each of the conditioned media, the fraction containing molecules greater that about 5 kDa supported hES cell growth when added to co-cultures of hES cells and non-supportive Hs27 cells. Fractionation of conditioned medium then was performed using a centrifugal filtration device having a NMWL of 30 kDa, thus retaining all molecules with a molecular weight greater than about 30 kDa. Like conditioned medium and the ≧5 kDa fraction, the ≧30 kDa fraction of CM from the transduced HAFI cells, and from pMEFs, supported hES cells cultured on the otherwise non-supportive Hs27 cells.

Although the invention has been described with reference to the above example, it will be understood that modifications and variations are encompassed within the spirit and scope of the invention. Accordingly, the invention is limited only by the following claims.

What is claimed is:

1. Isolated undifferentiated pluripotential human embryonic stem (hES) cells, wherein the hES cells exhibit dependence on adult human feeder cells, or an hES cell-maintaining product of said adult human feeder cells, for maintenance in culture.

2. The hES cells of claim 1, wherein the adult human feeder cells comprise human bone marrow cells.

3. The hES cells of claim 2, wherein the human bone marrow cells comprise human marrow stromal cells.

4. The hES cells of claim 1, wherein the adult human feeder cells comprise human fibroblasts.

5. The hES cells of claim 4, wherein the human fibroblasts comprise ATCC CCD-1087sk cells.

6. The hES cells of claim 1, wherein the adult human feeder cells comprise immortalized cells.

7. The hES cells of claim 6, wherein the immortalized cells contain an exogenous polynucleotide encoding a telomerase, which is expressed in said immortalized cells.

8. The hES cells of claim 1, wherein the product of said adult human feeder cells comprises conditioned medium.

9. The hES cells of claim 1, wherein the product of said adult human feeder cells comprises a fraction of conditioned medium comprising biomolecules having a molecular mass greater than about 30 kiloDaltons.

10. A culture of undifferentiated pluripotential human embryonic stem (hES) cells, comprising
hES cells, and
supportive adult human feeder cells, or an hES cell-maintaining product of said feeder cells.

11. The culture of claim 10, comprising the hES cells and the supportive adult human feeder cells.

12. The culture of claim 10, comprising the hES cells and the hES cell-maintaining product of the supportive adult human feeder cells.

13. The culture of claim 12, further comprising non-supportive feeder cells.

14. The culture of claim 13, wherein the non-supportive feeder cells are human cells.

15. The culture of claim 10, wherein the supportive adult human feeder cells comprise human bone marrow stromal cells or ATCC CCD-1087sk fibroblasts.

16. The culture of claim 10, wherein the hES cell-maintaining product of the supportive adult human feeder cells comprises conditioned medium, or a fraction of conditioned medium comprising biomolecules having a molecular mass greater than about 30 kiloDaltons.

17. A method of obtaining an expanded population of undifferentiated pluripotential human embryonic stem (hES) cells, comprising culturing hES cells, and supportive adult human feeder cells or an hES cell-maintaining product of said feeder cells, under conditions suitable for growth of the hES cells, thereby obtaining an expanded population of the hES cells.

18. A culture of undifferentiated pluripotential hES cells prepared by the method of claim 17.

19. The method of claim 17, comprising culturing the hES cells and the supportive adult human feeder cells.

20. The method of claim 17, wherein the supportive adult human feeder cells comprise human bone marrow stromal cells, ATCC CCD-1087sk fibroblasts, or a combination thereof.

21. The method of claim 17, wherein the supportive adult human feeder cells are immortalized.

22. The method of claim 17, comprising culturing the hES cells and the hES cell-maintaining product of the supportive adult human feeder cells.

23. The method of claim 22, further comprising culturing the hES cells and the supportive adult human feeder cells, or the hES cell-maintaining product of said feeder cells, with non-supportive feeder cells.

24. The method of claim 17, further comprising isolating hES cells of the expanded population of hES cells, thereby obtaining isolated undifferentiated pluripotential hES cells.

25. Isolated undifferentiated pluripotential hES cells obtained by the method of claim 24.

26. The method of claim 17, further comprising sub-culturing hES cells of the expanded population of hES cells under conditions suitable for growth, thereby obtaining a sub-culture of hES cells.

27. The method of claim 26, comprising sub-culturing the hES cells of the expanded population, and supportive adult human feeder cells or an hES cell-maintaining product of said feeder cells, under conditions suitable for growth of the hES cells.

28. The method of claim 26, further comprising repeating the sub-culturing step, thereby obtaining a continuous culture of undifferentiated pluripotential hES cells.

29. The method of claim 27, further comprising, before sub-culturing the hES cells, freezing an aliquot of the expanded population of hES cells.

30. The method of claim 29, wherein freezing the cells is performed under conditions such that the cells remain viable.

31. At least one aliquot of frozen undifferentiated pluripotent hES cells obtained by the method of claim 30.

32. A plurality of aliquots of frozen undifferentiated pluripotent hES cells of claim 31.

33. The plurality of claim 32, wherein aliquots of the plurality comprise hES cells having a predetermined passage number.

34. The method of claim 17, further comprising inducing differentiation of hES cells of the expanded population, thereby obtaining a population of differentiated cells.

35. A population of differentiated cells obtained by the method of claim 34.

36. A method for identifying an agent that alters a function of an undifferentiated pluripotential human embryonic stem (hES) cell, comprising:
a) contacting the hES cells with a test agent,
wherein the hES cells exhibit dependence on adult human feeder cells, or an hES cell-maintaining product of said adult human feeder cells, for maintenance in culture; and
b) detecting a change in a function of the hES cells in presence of the test agent as compared to the function in the absence of the test agent, thereby identifying the test agent as an agent that alters the fiction of the hES cells.

37. The method of claim 36, wherein said contacting is performed in vivo.

38. The method of claim 36, wherein said contacting is performed in vitro.

39. The method of claim 36, wherein the function of the hES cells is expression of stage-specific surface antigen-4 (SSEA-4), alkaline phosphatase, or Oct-4 transcription factor.

40. The method of claim 36, wherein the agent induces differentiation of the hES cells, thereby producing differentiated cells.

41. The method of claim 40, wherein the differentiated cells comprise multipotential human stem cells.

42. The method of claim 41, wherein the multipotential human stem cells comprise hematopoietic stem cells.

43. The method of claim 40, wherein the differentiated cells comprise terminally differentiated cells.

44. The method of claim 40, wherein the differentiated cells comprises muscle cells, neuronal cells, blood cells, connective tissue, or epithelial cells.

45. The method of claim 40, wherein the differentiated cells comprise pancreatic beta cells, hepatocytes, cardiomyocytes, or skeletal muscle cells.

46. A method of obtaining a cell culture medium for maintaining undifferentiated pluripotential human embryonic stem (hES) cell in culture, comprising:
a) culturing adult human cells that can support the growth of hES cells in culture; and
b) isolating conditioned medium generated by culturing the adult human cells, thereby obtaining a cell culture medium for maintaining undifferentiated pluripotential hES cells in culture.

47. The method of claim 46, wherein the adult human cells comprise human bone marrow stromal cells or ATCC CCD-1087sk fibroblasts.

48. The method of claim 46, further comprising isolating from the conditioned medium a fraction comprising biomolecules having a molecular mass greater than about 30 kiloDaltons.

49. The method of claim 48, wherein isolating the fraction comprises collecting a gel chromatography fraction.

50. Conditioned medium obtained by the method of claim 46.

51. Enriched hES cell growth factors, comprising a fraction of the conditioned medium of claim 50 comprising biomolecules having a molecular mass greater than about 30 kiloDaltons.

52. A method for obtaining undifferentiated pluripotential human embryonic stem (ES) cells, comprising:
a) culturing a suspension of cells comprising undifferentiated pluripotential hES cells, and supportive adult human feeder cells or an hES cell-maintaining product of said feeder cells, under conditions suitable for growth of the hES cells; and
b) isolating cells that express stage-specific surface antigen-4 (SSEA-4), Oct-4, and alkaline phosphatase, and do not express stage-specific surface antigen-1 (SSEA-1), thereby obtaining undifferentiated pluripotential hES cells.

53. The method of claim 52, comprising the hES cells and the supportive adult human feeder cells.

54. The method of claim 52, comprising the hES cells and the hES cell-maintaining product of the supportive adult human feeder cells.

55. The culture of claim 54, further comprising non-supportive feeder cells.

56. Isolated undifferentiated pluripotential hES cells obtained by the method of claim 52.

57. A method of ameliorating a pathologic condition in a subject, comprising administering undifferentiated pluripotential human embryonic stem (hES) cells, or cells derived from said hES cells, to the subject,
wherein the hES cells exhibit dependence on adult human feeder cells, or an hES cell-maintaining product of said adult human feeder cells, for maintenance in culture.

58. The method of claim 57, wherein the pathologic condition comprises a degenerative disorder.

59. The method of claim 58, wherein the degenerative disorder comprises Parkinson's disease, Alzheimer's disease, or muscular dystrophy.

60. The method of claim 57, wherein the pathologic condition comprises an autoimmune disorder.

61. The method of claim 60, wherein the autoimmune disorder is multiple sclerosis.

62. The method of claim 57, wherein the pathologic condition is diabetes.

63. The method of claim 57, wherein the pathologic condition comprises an injury.

64. The method of claim 63, wherein the injury comprises a spinal cord injury or a burn.


SEQUENCE LISTING (4 sequences)

SEQ ID NO: 1 - DNA, 24 bp, Artificial sequence
  Primer
cgtgmgctggagaaggagaagctg

SEQ ID NO: 2 - DNA, 25 bp, Artificial sequence
  Primer
caagggccgcagcttacacatgttc

SEQ ID NO: 3 - DNA, 21 bp, Artificial sequence
  Primer
gctcgtcgtcgacaacggctc

SEQ ID NO: 4 - DNA, 25 bp, Artificial sequence
  Primer
caaacatgatctgggtcatcttctc